(19) United States
(12) Patent Application Publication
(10) Pub. No.: US 2026/0053965 A1
(43) Pub. Date: Feb. 26, 2026

(54) Shoe Sanitization Device

(71) Applicants: Matthew Tucker, Rock Hill, SC (US); Stacy Childers, Rock Hill, SC (US)

(72) Inventors: Matthew Tucker, Rock Hill, SC (US); Stacy Childers, Rock Hill, SC (US)

(21) Appl. No.: 19/074,647

(22) Filed: Mar. 10, 2025

Related U.S. Application Data
(60) Provisional application No. 63/684,903, filed on Aug. 20, 2024.

(51) Int. Cl. A61L 2/10; A47L 23/02; A61L 2/20; A61L 2/26

(52) U.S. Cl. CPC A61L 2/10; A47L 23/02; A61L 2/202; A61L 2/26; A61L 2202/11; A61L 2202/14

(57) ABSTRACT

A shoe sanitization device is provided. The device is designed for the efficient and safe disinfection and deodorization of shoe soles, suitable for residential, commercial, and institutional use. The device comprises a durable, antimicrobial housing resembling a bathroom scale, featuring a textured non-slip surface for stability. The device employs UV-C light technology with a wavelength range of 200 to 280 nanometers and reflective surfaces for uniform pathogen neutralization, activated automatically by pressure sensors. Additional safety measures include sensors preventing accidental UV exposure. The device is powered by a rechargeable battery and incorporates LED indicators for operational status and a deodorization module using ozone or activated carbon filters.

### CROSS-REFERENCE TO RELATED APPLICATION

The present application claims priority to, and the benefit of, U.S. Provisional Application No. 63/684,903 which was filed on Aug. 20, 2024, and is incorporated herein by reference in its entirety.

### FIELD OF THE INVENTION

The present invention relates generally to the field of sanitization devices. More specifically, the present invention relates to a device that efficiently disinfects and deodorizes shoe soles using UV-C light technology and optional ozone or activated carbon filters. Accordingly, the present disclosure makes specific reference thereto. Nonetheless, it is to be appreciated that aspects of the present invention are also equally applicable to other like applications, devices, and methods of manufacture.

### BACKGROUND

Footwear is a significant source of germs and bacteria accumulation, posing a substantial public health challenge in both private and public environments. Unlike clothing items that are regularly cleaned, shoes are often neglected in hygiene routines, allowing contaminants to persist on their surfaces. These pathogens can be unknowingly spread to various locations, including hospitals, schools, airports, and homes, increasing the risk of illnesses caused by bacteria, viruses, and fungi. High-traffic areas are especially susceptible to contamination due to the volume of individuals wearing unsanitized footwear. Efforts to maintain cleanliness are further complicated by the complex design of shoe soles, which feature ridges and grooves that harbor microbes. Traditional cleaning methods for shoes are time-consuming, inefficient, and impractical for frequent use. This problem underscores the necessity of an effective and user-friendly solution to disinfect footwear, particularly in environments where maintaining strict hygiene protocols is critical.

Therefore, there exists a long-felt need in the art for a shoe sanitization device that eliminates germs and bacteria effectively from the soles of shoes. There also exists a long-felt need in the art for a shoe sanitization device that operates quickly and conveniently, enabling frequent use without significant time investment. Moreover, there exists a long-felt need in the art for a shoe sanitization device that incorporates safety features to prevent accidental activation and ensures reliable operation in diverse environments.

The subject matter disclosed and claimed herein, in one embodiment thereof, comprises a shoe sanitization device. The device is designed for the efficient and safe disinfection and deodorization of shoe soles, suitable for residential, commercial, and institutional use. The device comprises a durable, antimicrobial housing resembling a bathroom scale, featuring a textured non-slip surface for stability. The device employs UV-C light technology with a wavelength range of 200 to 280 nanometers and reflective surfaces for uniform pathogen neutralization, activated automatically by pressure sensors. Additional safety measures include sensors preventing accidental UV exposure. The device is powered by a rechargeable battery and incorporates LED indicators for operational status and a deodorization module using ozone or activated carbon filters.

In this manner, the shoe sanitization device of the present invention accomplishes all the foregoing objectives and provides a footwear disinfection device that utilizes integrated UV-C lights capable of neutralizing a broad spectrum of pathogens. The pressure sensors ensure ease of use by activating the sanitization cycle when a user standing on the device is detected. The inclusion of non-slip surfaces and shatterproof materials enhances user safety and durability. Reflective surfaces within the device ensure even light distribution, effectively disinfecting grooves and ridges on shoe soles. Additionally, the device is equipped with LED indicators for operational feedback and optional deodorization modules to neutralize odors, promoting a comprehensive sanitization process. The device's versatile functionality, accommodating both battery-powered and wired configurations, ensures applicability across residential, commercial, and institutional settings, supporting hygiene and reducing the spread of illness in high-traffic environments.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a shoe sanitization device designed to disinfect and deodorize shoe soles for use in residential, commercial, and institutional settings. The device comprises a durable, antimicrobial housing, available in various shapes and constructed from materials resistant to microbial growth. A textured, non-slip top surface enhances user safety, utilizing materials such as rubber or thermoplastic elastomers.

The device incorporates at least one UV-C light beneath a transparent, shatterproof top surface, emitting wavelengths between 200-280 nanometers to neutralize pathogens. Reflective surfaces within the housing ensure uniform light distribution. The UV-C light operates on an adjustable cycle and is activated by pressure sensors detecting footwear presence. Additional safety sensors prevent accidental activation and exposure to non-footwear objects.

Power is supplied via a rechargeable lithium-ion battery or wired connection, supporting multiple disinfection cycles. A user interface with LED indicators provides real-time status updates, signaling readiness, active cycles, or maintenance requirements.

An optional deodorization module, comprising a mild ozone generator or activated carbon filter, neutralizes odors. The module is removable and replaceable for ease of maintenance.

The device is suitable for various applications, including preventing contaminants in homes, supporting hygiene protocols in public and institutional spaces, and providing a portable solution for travelers. It offers benefits such as reduced pathogen transmission, ease of use, and adaptability to different environments.

The method of use involves stepping onto the non-slip surface, activating the UV-C light and optional deodorization module via sensors, and completing a disinfection cycle. LED indicators signal process completion, ensuring user

Accordingly, the shoe sanitization device of the present invention is particularly advantageous as it provides a footwear disinfection device that utilizes integrated UV-C lights capable of neutralizing a broad spectrum of pathogens. The pressure sensors ensure ease of use by activating the sanitization cycle when a user standing on the device is detected. The inclusion of non-slip surfaces and shatterproof materials enhances user safety and durability. Reflective surfaces within the device ensure even light distribution, effectively disinfecting grooves and ridges on shoe soles. Additionally, the device is equipped with LED indicators for operational feedback and optional deodorization modules to neutralize odors, promoting a comprehensive sanitization process. The device's versatile functionality, accommodating both battery-powered and wired configurations, ensures applicability across residential, commercial, and institutional settings, supporting hygiene and reducing the spread of illness in high-traffic environments.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a shoe sanitization device of the present invention in accordance with the disclosed architecture; and

FIG. 2 illustrates a flowchart of a method of using one potential embodiment of a shoe sanitization device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a shoe sanitization device that eliminates germs and bacteria effectively from the soles of shoes. There also exists a long-felt need in the art for a shoe sanitization device that operates quickly and conveniently, enabling frequent use without significant time investment. Moreover, there exists a long-felt need in the art for a shoe sanitization device that incorporates safety features to prevent accidental activation and ensures reliable operation in diverse environments.

The present invention, in one exemplary embodiment, is comprised of a shoe sanitization device designed to disinfect and deodorize shoe soles for residential, commercial, and institutional use. The device is comprised of a durable, antimicrobial housing in various shapes, made from materials resistant to microbial growth. A textured, non-slip top surface enhances safety and is constructed from materials such as rubber or thermoplastic elastomers.

The device includes at least one UV-C light positioned beneath a transparent, shatterproof top surface, emitting wavelengths between 200-280 nanometers to neutralize pathogens. Reflective surfaces within the housing ensure uniform light distribution. The UV-C light operates on an adjustable cycle and is activated by pressure sensors detecting footwear presence, with additional safety sensors preventing accidental activation or exposure to non-footwear objects.

Power is supplied via a rechargeable lithium-ion battery or wired connection, enabling multiple disinfection cycles. A user interface with LED indicators provides real-time status updates, signaling readiness, active cycles, and maintenance requirements.

An optional deodorization module, comprised of a mild ozone generator or activated carbon filter, neutralizes odors and is designed to be removable and replaceable for maintenance convenience.

The device is suitable for various applications, including minimizing contaminants in homes, supporting hygiene protocols in public and institutional spaces, and serving as a portable solution for travelers. Benefits include reduced pathogen transmission, user-friendliness, and adaptability to different environments.

The method of use involves stepping onto the non-slip surface, activating the UV-C light and optional deodorization module via sensors, and completing a disinfection cycle. LED indicators provide feedback on process completion.

The shoe sanitization device offers significant advantages by integrating UV-C lights capable of neutralizing a wide range of pathogens. Pressure sensors ensure ease of operation by activating the sanitization cycle upon detecting footwear. Non-slip surfaces and shatterproof materials enhance safety and durability, while reflective surfaces promote even light distribution for effective disinfection of grooves and ridges. LED indicators provide operational feedback, and optional deodorization modules neutralize odors, enabling a comprehensive sanitization process. The device's versatility, with battery-powered and wired configurations, makes it applicable across residential, commercial, and institutional environments, supporting hygiene and reducing illness spread in high-traffic areas.

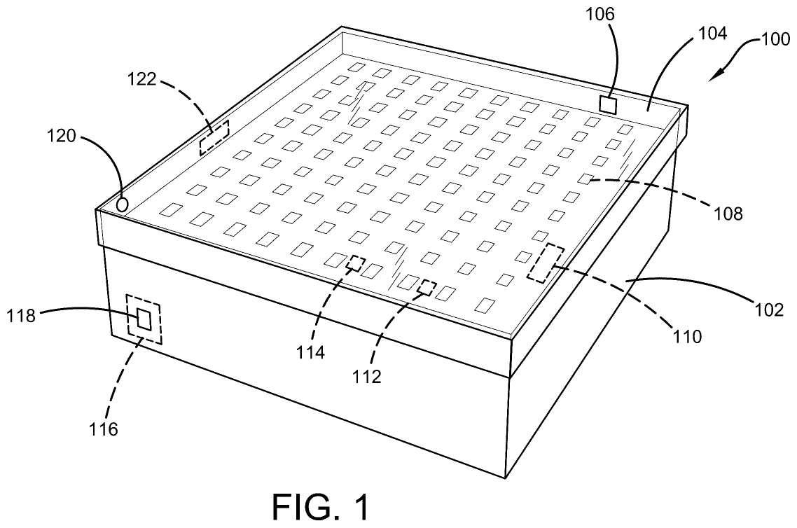

Referring initially to the drawings, FIG. 1 illustrates a perspective view of one potential embodiment of a shoe sanitization device 100 of the present invention in accordance with the disclosed architecture. The present invention relates to a shoe sanitization device 100 designed to disinfect and deodorize the soles of shoes efficiently and safely. The device 100 is particularly suited for use in residential, commercial, and institutional environments where maintaining hygiene is a priority.

The shoe sanitization device 100 is comprised of a box-like housing 102 resembling the form factor of a bathroom scale. However, the housing 102 may be any shape, such as, but not limited to, rectangular, circular, or oval, allowing customization to suit various aesthetic and functional preferences. The housing 102 is preferably constructed from durable, antimicrobial materials, such as high-grade plastics or metals, ensuring long-term resilience and maintaining a hygienic surface that resists microbial growth. A top surface 104 of the housing 102 is comprised of a textured, non-slip area 106 that provides stability and safety when users step onto the device 100. The texture may include, but is not limited to, ridged, grooved, or pebble-like patterns designed to prevent slippage. The material of the non-slip area 106 may be any suitable non-slip material, such as, but not limited to, rubber, thermoplastic elastomers, or textured composites, further enhancing user safety.

The housing 102 is further comprised of at least one UV-C light 108 housed beneath the top surface 104. The top surface 104 is preferably transparent and shatterproof, serving to protect the light 108 while allowing effective light transmission. The UV-C light 108 emits a high-intensity wavelength, such as, but not limited to, wavelengths between 100 and 480 nanometers, capable of neutralizing a broad spectrum of pathogens, including bacteria, viruses, and fungi. In different embodiments, the UV-C light 108 may be different UV light types such as but not limited to UV-A, UV-B, etc. To enhance the effectiveness of the UV-C light 108, the housing 102 may be comprised of at least one reflective surface 110 strategically positioned at specific angles within the housing 102. These reflective surfaces 110 ensure uniform light distribution across the shoe soles, effectively reaching grooves, ridges, and other areas where contaminants are likely to accumulate. The disinfection process of the UV-C light 108 preferably operates on a pre-programmed cycle adjustable between 10 and 30 seconds. The duration of the cycle is optimized to achieve complete disinfection while balancing energy efficiency and user convenience.

The activation of the UV-C light 108 is controlled by at least one sensor 112. The sensor 112 is preferably a pressure sensor embedded beneath the top surface 104, designed to detect the presence of a user standing on the top surface 104. The pressure sensor 112 automatically initiates the disinfection cycle of the light 108 when the weight threshold associated with typical footwear is detected. To further enhance safety, the device 100 may be comprised of additional sensors 114 that prevent accidental activation of the UV-C lights 108. More specifically, the sensor 114 may be comprised of an optical or infrared safety sensor that detects non-footwear objects, such as hands, tools, or other items inadvertently placed on the device 100. Upon detecting such objects, the safety sensor 114 immediately deactivates the light 108 to prevent harmful exposure. These additional sensors 114 may also include proximity sensors to enhance responsiveness and prevent unintended activation from objects near the device 100.

Power is supplied to the shoe sanitization device 100 through a rechargeable lithium-ion battery 116. The battery 116 may be disposable or rechargeable via a charging port 118, such as, but not limited to, a USB-C or magnetic charging interface, enabling quick and convenient recharging. The battery 116 is capable of supporting multiple disinfection cycles per charge, with the specific number dependent on the operational duration of each cycle. For environments with high foot traffic, a wired power mode is also available, allowing continuous operation without reliance on the battery 116. This dual-power functionality ensures versatility for various applications, from portable use to permanent installation in commercial settings.

The device 100 includes a user interface featuring a system of LED indicators 120 to communicate operational status. An LED 120 that illuminates a first color, such as green, signals that the device 100 is ready for use. An LED 120 of a second color, such as blue, illuminates during an active disinfection cycle, providing clear feedback to the user. An LED 120 of a third color, such as red, alerts users to safety concerns or maintenance needs, such as depleted battery charge, filter replacement, or the need for UV-C light 108 replacement. These indicators ensure the user remains informed about the device's status at all times.

In addition to its disinfection capabilities, the shoe sanitization device 100 is optionally equipped with a deodorization module 122, as seen in FIG. 1. This module 122 may be implemented in two configurations: a mild ozone generator or an activated carbon filter. The ozone generator releases controlled amounts of ozone into the enclosed area beneath the top surface 104. Ozone acts as an antimicrobial agent, eliminating odors and further reducing microbial presence. The activated carbon filter, on the other hand, absorbs volatile organic compounds responsible for unpleasant odors, leaving shoes with a fresh and neutral scent after the sanitization process. The deodorization module 122 is designed for easy maintenance and is preferably removable and replaceable.

The shoe sanitization device 100 is suitable for a variety of applications. In residential settings, the device 100 prevents the introduction of outdoor contaminants into living spaces, promoting a cleaner and more hygienic home environment. In commercial and institutional environments, such as hospitals, schools, and retail establishments, the device 100 supports hygiene protocols by sanitizing footwear at entry points, reducing the spread of pathogens. Its compact and lightweight design also makes it ideal for travelers, offering a portable solution for maintaining shoe hygiene on the go.

The shoe sanitization device 100 provides numerous benefits, including the reduction of bacteria, germs, and viruses on shoe soles, thereby promoting public and personal health. Its automatic operation, intuitive interface, and safety features make it convenient and user-friendly. The device's versatile design ensures its effectiveness in a wide range of settings, from private homes to high-traffic public spaces.

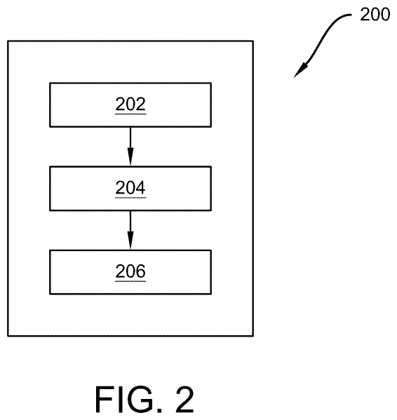

The present invention is also comprised of a method of using 200 the shoe sanitization device 100, as seen in FIG. 2. First, a device 100 is provided comprised of a housing 102, at least one UV-C light 108, at least one sensor 112, and optionally a deodorization module 122 [Step 202]. Next, a user steps onto the top surface 104 of the housing 102, ensuring their shoes are in direct contact with the non-slip area 106 and/or places their shoes on the top surface 104 [Step 204]. The sensor 112 detects the presence of footwear and automatically activates the UV-C light 108 to initiate the disinfection cycle. Then, the UV-C light 108 emits a high-intensity wavelength, while reflective surfaces 110 ensure uniform light distribution across the soles of the footwear. During this process, the deodorization module 122, if present, neutralizes odors using a mild ozone generator or an activated carbon filter. Then, upon completion of the pre-programmed disinfection cycle, the UV-C light 108 automatically deactivates, and the LED indicators 120 signal the end of the process, indicating the footwear has been sanitized and is safe for use. Finally, a user steps off of the device 100 with sanitized shoes [Step 206].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “shoe sanitization device” and “device” are interchangeable and refer to the shoe sanitization device 100 of the present invention.

Notwithstanding the foregoing, the shoe sanitization device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the shoe sanitization device 100 as shown in the FIGS. are for illustrative purposes only, and that many other sizes and shapes of the shoe sanitization device 100 are well within the scope of the present disclosure. Although the dimensions of the shoe sanitization device 100 are important design parameters for user convenience, the shoe sanitization device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A shoe sanitization device comprising:
a housing having a top surface, the top surface comprised of a non-slip area;
a UV-C light positioned beneath the top surface;
a reflective surface disposed within the housing, the reflective surface configured to direct a light emitted by the UV-C light uniformly across a shoe sole placed on the top surface;
a sensor configured to detect the presence of a user on the top surface and wherein activation of the sensor activates the UV-C light in response; and
a power source configured to supply power to the UV-C light.

2. The shoe sanitization device of claim 1, wherein the top surface is comprised of a transparent material to allow light transmission from the UV-Light to reach the shoe sole.

3. The shoe sanitization device of claim 1, wherein the UV-C light emits a wavelength between 200 and 280 nanometers.

4. The shoe sanitization device of claim 1, wherein the sensor is comprised of a pressure sensor.

5. The shoe sanitization device of claim 1 further comprising a charging port.

6. The shoe sanitization device of claim 1 further comprising a safety sensor that deactivates the UV-C light.

7. The shoe sanitization device of claim 1, wherein the non-slip area is comprised of a texture.

8. A shoe sanitization device comprising:
a housing having a top surface, the top surface comprised of a non-slip area;
a UV-C light positioned beneath the top surface;
an LED indicator;
a reflective surface disposed within the housing, the reflective surface configured to direct a light emitted by the UV-C light uniformly across a shoe sole placed on the top surface;
a sensor configured to detect the presence of a user on the top surface and wherein activation of the sensor activates the UV-C light in response; and
a power source configured to supply power to the UV-C light.

9. The shoe sanitization device of claim 8, wherein the LED indicator displays a first color to indicate the shoe sanitization device is ready to use.

10. The shoe sanitization device of claim 8, wherein the LED indicator displays a second color to indicate the shoe sanitization device is actively disinfecting.

11. The shoe sanitization device of claim 8, wherein the LED indicator displays a third color to indicate a safety alert or a maintenance alert.

12. The shoe sanitization device of claim 8 further comprised of a safety sensor.

13. The shoe sanitization device of claim 12, wherein the safety sensor is comprised of an optical sensor.

14. The shoe sanitization device of claim 12, wherein the safety sensor is comprised of a proximity sensor.

15. The shoe sanitization device of claim 8 further comprised of a deodorization module.

16. The shoe sanitization device of claim 15, wherein the deodorization module is comprised of an ozone generator.

17. The shoe sanitization device of claim 15, wherein the deodorization module is comprised of an activated carbon filter.

18. The shoe sanitization device of claim 8, wherein the power source is comprised of a battery.

19. The shoe sanitization device of claim 18, wherein the battery is comprised of a charging port.

20. A method of using a shoe sanitization device, the method comprising the following steps:
providing a shoe sanitization device comprising a housing with a top surface, a UV-C light positioned beneath the top surface, and a sensor configured to detect the presence of a user standing on the shoe sanitization device;
placing footwear on the top surface of the housing;
viewing an LED indicator to indicate the footwear has been sanitized; and
removing the footwear from the top surface of the housing.
